(19) United States
(12) Patent Application Publication
(10) Pub. No.: US 2008/0200439 A1
(43) Pub. Date: Aug. 21, 2008

(54) Methods and compositions for modulating BK channel activity and vasodilation

(75) Inventors: Alejandro M. Dopico, Cordova, TN (US); Anna Nodarovna Bukiya, Memphis, TN (US); Jianxi Liu, Memphis, TN (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: University of Tennessee Research Foundation, Memphis, TN (US)

(21) Appl. No.: 12/005,639

(22) Filed: Dec. 27, 2007

Related U.S. Application Data
(60) Provisional application No. 60/877,295, filed on Dec. 27, 2006.

(51) Int. Cl. A61K 31/575; C12N 9/00; A61P 9/00

(52) U.S. Cl. 514/169; 435/4

(57) ABSTRACT

Methods and compositions are provided for modulating myocyte BK channel activity and for screening for modulators of BK channel activity. The methods and compositions are useful in the treatment of disorders where increasing myocyte BK channel activity can attenuate, revert or prevent the disorder. For example, the methods and compositions are useful for treating disorders ameliorated by increasing vasodilation and blood flow in a subject, in particular, increasing cerebral blood flow.

### RELATED APPLICATIONS

This application claims priority to U.S. Provisional Application Ser. No. 60/877,295, filed Dec. 27, 2006, herein incorporated by reference in its entirety.

### GOVERNMENT INTEREST

The presently disclosed subject matter was made with United States Government support under Grant Nos. HL77424 and AA11560 awarded by the National Institutes of Health. Accordingly, the United States Government has certain rights in the presently disclosed subject matter.

### TECHNICAL FIELD

The presently disclosed subject matter relates to methods and compositions for modulating BK channels and vasodilation and methods of treating disorders of vasodilation, in particular, methods for increasing cerebral blood flow.

### TABLE OF ABBREVIATIONS

LC lithocholate
BK large-conductance Ca²⁺-activated K⁺ channel
PSS physiological saline solution
Ibtx iberiotoxin
DM dissociation medium
I/O inside-out
O/O outside-out
N number of functional channels in the patch
Pₒ open channel probability
DMSO dimethyl sulfoxide
CMC critical micellar concentration
CBF cerebral blood flow
K_{V} voltage-gated K⁺ channel
4-AP 4-aminopyridine
Z effective valence
DHS-1 dehydrosoyasaponin-1

### BACKGROUND

Cerebrovascular vasodilators are of particular interest considering that 1) stroke remains the third leading cause of death and first cause of long-term disability in the United States; 2) greater than 88% of strokes are ischemic (Williams et al., 2003), in which impaired vasomotion may be found; and 3) biomedical research has largely failed to provide effective and safe cerebrovascular dilators (Legos et al., 2002).

Accordingly, there is a need for safer and more effective vasodilators. The presently disclosed subject matter provides methods and compositions for modulating large-conductance, Ca²⁺-activated K⁺ (BK) channels and vasodilation and, in particular, vasodilation of small resistance arteries.

### SUMMARY

The presently disclosed subject matter provides methods and compositions for modulating myocyte large-conductance, Ca²⁺-activated K⁺ (BK) channel activity mediated through the β₁ subunit and for affecting vasodilation. In some embodiments, methods are provided for screening candidate compositions for an ability to modulate myocyte BK channel activity, comprising, establishing a test sample comprising a myocyte BK channel β₁ subunit, and measuring the interaction, effect, or combination thereof, of a candidate composition on the test sample to thereby determine the ability of the candidate composition to modulate myocyte BK channel activity. In some embodiments, the candidate composition comprises a lithocholate, or an analog or derivative thereof. In some embodiments, the myocyte is a small resistance artery myocyte.

In some embodiments, the presently disclosed subject matter provides methods of screening for candidate compositions useful in the treatment of a disorder where increasing myocyte BK channel activity can attenuate, revert or prevent the disorder, comprising, establishing a test sample comprising a myocyte BK channel β₁ subunit, and measuring the interaction, effect, or combination thereof, of a candidate composition on the test sample to thereby determine the ability of the candidate composition to increase myocyte BK channel activity and attenuate, revert or prevent the disorder. In some embodiments, the candidate composition comprises a lithocholate, or an analog or derivative thereof. In some embodiments, the myocyte is a small resistance artery myocyte. In some embodiments, the attenuation, reversion or prevention of the disorder is mediated at least in part through modulation of blood pressure and/or blood flow. In some embodiments, the attenuation, reversion or prevention of the disorder is mediated at least in part through vasodilation. In some embodiments, vasodilation is characterized by an increase in blood vessel diameter of about 10 percent.

In some embodiments, the presently disclosed subject matter provides methods of treating a subject having a disorder, comprising, administering to the subject a composition comprising a compound capable of increasing myocyte BK channel activity mediated by the BK channel β₁ subunit, wherein increasing myocyte BK channel activity can attenuate, revert or prevent the disorder. In some embodiments, the compound comprises a lithocholate, or an analog or derivative thereof. In some embodiments, the administration of the composition comprising the compound capable of increasing myocyte BK channel activity results in decreases in blood pressure and/or increases in blood flow. In some embodiments, the increase in blood flow is mediated at least in part through vasodilation. In some embodiments, the vasodilation occurs at least in part in small resistance arteries. In some embodiments, the small resistance arteries are small cerebral arteries. In some embodiments, the vasodilation in the small cerebral arteries is about a 10 percent increase in arterial diameter. In some embodiments, the increase in blood flow in the small cerebral arteries is about 30 percent.

In some embodiments, the presently disclosed subject matter provides a pharmaceutical composition useful for increasing myocyte BK channel activity in a subject, wherein the composition comprises an effective amount of a lithocholate or an analog or derivative thereof, and a pharmaceutically acceptable carrier. In some embodiments, pharmaceutical compositions are provided that are useful for increasing myocyte BK channel activity in a subject, wherein the BK channel activity is mediated by the BK channel β₁ subunit.

In some embodiments, the presently disclosed subject matter provides methods of selectively targeting tissues comprising BK channel β₁ subunits in a subject, comprising, administering to the subject a composition comprising a compound capable of selectively binding myocyte BK channel β₁ subunits, whereby tissues comprising BK channel β₁ subunits are targeted. In some embodiments, the composition comprises a lithocholate, or an analog or derivative thereof. In some embodiments, the tissues targeted are smooth muscle tissues. In some embodiments, the composition further comprises an imaging agent. In some embodiments, the composition further comprises a pharmaceutically acceptable carrier. In some embodiments, the subject is a mammal.

Accordingly, it is an object of the presently disclosed subject matter to provide methods and compositions for modulating BK channels and vasodilation. This and other objects are achieved in whole or in part by the presently disclosed subject matter.

An object of the presently disclosed subject matter having been stated above, other objects and advantages will become apparent upon a review of the following descriptions, figures and examples.

### BRIEF DESCRIPTION OF THE DRAWINGS

FIGS. 1A-1D show lithocholate (LC) dilates pressurized arteries via activation of BK channels independently of an intact endothelium.

FIG. 1A is a rat cerebral arterial diameter trace showing that after artery development of myogenic tone, acute application of 45 μM LC causes sustained yet fully reversible dilation. LC action is practically abolished by 55 nM iberiotoxin (Ibtx), a selective BK channel blocker (the vasodilatory “rebound” following Ibtx wash is due to increased flow rate; see Materials and Methods).

FIG. 1B is a diameter trace showing that LC-induced dilation is unaffected by 0.8 mM 4-aminopyridine (4-AP), a blocker of K_{V} channels other than BK. In A) and B), vertical dotted lines indicate the times at which arterial diameter was determined.

FIG. 1C is a plot showing averaged diameter in response to LC (n=17), Ibtx+LC (n=4), and 4-AP+LC (n=3). LC specific action on diameter is highlighted by displaying data as a percent change from values obtained in vehicle with (second and third column) or without (first column) K⁺ channel blockers.

FIG. 1D is a plot showing LC-induced dilation is similar in intact vs. endothelium-denuded arteries (n=5). The presence of a functional endothelium was assessed by responses to endothelium-dependent (10 μM acetylcholine {Ach}; n=4) and independent (10 μM sodium nitroprusside {SNP}; n=5) vasodilators. **Different from intact arteries (p<0.01).

FIGS. 2A-2B show LC at submillimolar concentrations activates native BK channels in freshly isolated rat cerebral artery myocytes.

FIG. 2A is a set of single-channel recordings from an I/O patch excised from an arterial myocyte before, during, and after 45 μM LC. Vehicle-containing solution was applied before (Vhcl) and after (Washout) LC-containing solution. Openings are shown as upward deflections; arrows indicate the baseline; Vm=−40 mV, free Ca²⁺_{i≈}3 μM.

FIG. 2B is a graph showing LC action is concentration-dependent: EC₅₀=46±6 μM; Eₘₐₓ≈3000 μM, at which NPₒ reaches ˜350% of control (n≧3).

FIGS. 3A-3F show β₁, but not β4 subunits, confer LC sensitivity to BK channels.

FIG. 3A is a set of records from I/O patches showing that 150 μM LC fails to increase homomeric cbv1 (i.e., rslo1) (Vm=−20 mV, free Ca²⁺ᵢ≈10 μM).

FIG. 3B is a set of records from I/O patches showing that, in contrast, LC enhances heteromeric cbv1+β₁ NPₒ under identical conditions (Vm=−20 mV, free Ca²⁺ᵢ≈10 μM).

FIG. 3C is a graph showing while LC fails to potentiate cbv1 channels even at 300 μM, LC activates cbv1+β₁ channels in a concentration-dependent manner: Eₘₐₓ≈300 μM; EC₅₀=43.5±4.7 μM. These values are almost identical to those obtained with native channels in rat cerebral artery myocytes (see FIG. 2).

FIG. 3D is a logit-log plot of LC action on cbv1+β₁ showing data fitted to a sigmoidal function, which renders a slope=1.32. To construct this plot, Eₘₐₓ was calculated as the mean of NPₒ values obtained at 150 and 300 μM LC.

FIG. 3E is a set of records from I/O patches showing that LC at concentrations that maximally activate native BK and cbv1+β₁ channels fails to activate cbv1+β₄ channels.

FIG. 3F is a graph showing the averaged ratios of NPₒ in the presence (NP_{o LC}) and absence (NP_{o Vhcl}) of 150 μM LC for cbv1 (n=6), cbv1+β₁ (n=6), and cbv1+β₄ (n=9) channels expressed in Xenopus oocytes. **Different from cbv1+β₁ (p<0.01).

FIGS. 4A-4B show LC increases BK unitary currents by increasing Pₒ, which increase is secondary to a mild increase in mean open time and a marked decrease in mean closed time.

FIG. 4A is a set of current records from an I/O patch containing a single cbv1+β₁ channel expressed in Xenopus oocytes in the absence (left) and presence (right) of 150 μM LC. The LC increase in Pₒ (=320%) is similar to the LC increase in NPₒ (≈290±45%; see FIG. 3C, FIG. 4B and Example 3), indicating that LC action occurs without an increase in the number of channels (N); Vₘ set to +20 mV; free Ca²⁺₁≈10 μM. Arrows on the left of the top traces of each panel indicate the baseline, and channel openings are shown as downward reflections.

FIG. 4B is a table showing cbv1+β₁ channel dwell-times in the absence and presence of 150 μM LC. Both open and closed time distributions could be well fitted with a double-exponential function, indicating the existence of at least two open (fast and slow) and two closed (fast and slow) states. The table shows both the average duration of each component (T) and its contribution to the total time spent in open (closed) states (as percentage in parentheses). The LC increase in Pₒ (˜320%) is caused by a mild increase in the average duration of both short and long open events and a sharp decrease (−60%) in mean close time, the latter basically due to LC-induced destabilization of channel long closures.

FIGS. 5A-5B show LC activation of BK channels within the physiological ranges of [Ca^{2+]}ᵢ and membrane potential.

FIG. 5A is a graph showing NPₒ during exposure of the intracellular side of I/O patches to 150 μM LC(NP_{o LC}) vs. NPₒ in vehicle-containing solution (NP_{o Vhcl}) plotted as a function of free [Ca^{2+]}ᵢ. Channel NPₒ was obtained following coexpression of cbv1 and β₁-subunits in Xenopus oocytes. The membrane voltage was set within the range ±20 mV, and the bath solution contained 0.1 (n=4), 0.3 (n=3), 1 (n=3), 3 (n=7), 10 (n=6) or 30 μM (n=4) free [Ca²⁺]ᵢ. Each column represents the mean ±SEM.

FIG. 5B is a graph showing the voltage needed for half-maximal increase in BK channel NPₒ (V_{0.5}) as a function of [Ca²⁺]ᵢ in the vehicle-containing solution (Vhcl) and in the presence of 150 μM LC. V_{0.5} values were obtained from G/Gₘₐₓ curves for I/O macropatches at 0.3, 3, and 10 μM Ca²⁺ᵢ. Voltage steps 200 msec duration were applied from −150 to +150 mV with 10-mV increments, V_{holding}=0 mV. Each data point represents the mean value ±SEM from ≧4 patches (oocytes). At every [Ca²⁺]ᵢ LC causes a similar leftward shift in V_{0.5} of ˜17.7 mV.

FIGS. 6A-6B show LC failure to dilate pressurized arteries from β₁-subunit knockout mice.

FIG. 6A is a set of arterial diameter traces showing that acute 45 μM LC and 55 nM Ibtx cause sustained diameter increase and decrease, respectively, in arteries from wt mice (grey trace) but not in arteries from BK β₁-knockout mice (KCNMB1-knockout mice) (black trace). The small and transient dilation caused by vehicle (Vhcl) is similar in both mice.

FIG. 6B is a graph showing averaged diameter data in response to LC (left) and Ibtx (right) in wt (hollow) (n=7) and β₁-knockout (black bars) (n=5) mice. *Different from wt mice (p<0.05); **Different from wt mice (p<0.01).

FIGS. 7A-7B show coexpression of β₁-subunits modifies both current kinetics and pharmacology of cbv1 channels.

FIG. 7A is a set of current records from an outside out (O/O) patch containing a single cbv1 channel (top) or cbv1+β₁ channel (bottom) expressed in Xenopus oocytes. Currents with fast activation kinetics are shown for the Xenopus oocytes expressing cbv1 channels (Tᵣᵢₛₑ=0.86±0.07 msec at Vₛₜₑₚ=+80 mV) (top). Co-expression of β₁-subunits (bottom) slows down current activation (Tᵣᵢₛₑ=4.24±0.17 msec at Vₛₜₑₚ=+80 mV). β₁-subunits also increase total current at any given voltage.

FIG. 7B is a graph showing β₁-subunits render BK channels sensitive to low micromolar concentrations of 17β-estradiol. The graph shows averaged potentiation in channel activity by 10 μM 17β-estradiol (n=4).

FIGS. 8A-8B show coexpression of β₄-subunits modifies the phenotype of cbv1 channels.

FIG. 8A is a graph showing the averaged ratios of NPₒ for cbv1 and cbv1+β₄ channels expressed in Xenopus oocytes in the presence of 55 nM iberiotoxin (NP_{o Iberiotoxin}) and absence of iberiotoxin (NP_{o Cnt}). The data indicate β₄-subunits introduce resistance to block by protracted exposure to iberiotoxin (n=3-6). *Significantly different from values in cbv1+β₄ channels, p<0.05; **Significantly different from values in cbv1+β₄, p<0.01.

FIG. 8B is a graph showing the averaged V_{0.5} for cbv1 (n=3), cbv1+β₁ (n=3), and cbv1+β₄ (n=5) channels expressed in Xenopus oocytes. When coexpressed with α-subunits, β₄-subunits introduce a hyperpolarizing shift in V_{0.5} similar to that caused by α+β₁ coexpression. V_{0.5} values were obtained from G/Gₘₐₓ plots fitted to Boltzmann functions. *Significantly different from cbv1 channels, p<0.05.

### DETAILED DESCRIPTION

The presently disclosed subject matter provides methods and compositions for modulating myocyte BK channel function. In some embodiments, the compositions of the presently disclosed subject matter comprise lithocholate (LC) and LC analogs and derivatives (LC-like compounds) that selectively target myocyte BK channel function and are effective dilators of pressurized resistance arteries. Accordingly, methods and compositions are provided herein comprising LC and LC-like compounds for modulating myocyte BK channel function and vasodilation, for screening for modulators of myocyte BK channel function, and for use in treatment methods.

### I. DEFINITIONS

While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a carrier” includes a plurality of such carriers, and so forth.

The term “about”, as used herein when referring to a measurable value such as an amount of weight, time, etc. is meant to encompass variations of, in some embodiments ±20% or ±10%, in some embodiments ±5%, in some embodiments ±1%, in some embodiments ±0.5%, and in some embodiments ±0.1%, from the specified amount, as such variations are appropriate to perform the disclosed methods.

As used herein, the term “analog” refers to a compound having structural similarity to the compound or molecule for which it is an analog. In some embodiments, an analog can be prepared from a compound that has been modified by deletion, addition, modification or substitution of one or more chemical moieties.

As used herein, the term “bile acid” refers to any of the large group of steroids derived from cholanic acid. Naturally occurring bile acids are mainly produced in the liver of mammals through the oxidation of cholesterol. For these bile acids, the joint between steroidal rings A and B is usually in cis configuration (i.e., C5 in β configuration). Thus, these naturally occurring bile acids derive from 5β-cholanic acid (i.e., 5β-cholan-24-oic acid). These bile acids are stored in the gallbladder and are released into the intestine lumen to help to absorb fat. Further, these naturally occurring bile acids have been claimed to serve as endogenous, ileal vasodilators. Bile acid derivatives can have the joint between steroidal rings A and B in trans configuration (i.e., C5 in a configuration). These bile acids derive from 5α-cholanic acid (i.e., 5α-cholan-24-oic acid) and are collectively refer to as “allo” bile acids. Bile acids commonly used in the presently disclosed subject matter, whether naturally occurring or synthetic, include cholic acid, deoxycholic acid, taurolithocholic acid, lithocholic acid, cholic acid methyl ester, cholic alcohol, 7,12 deoxycholic acid, lithocholic acid 3-hemisuccinate, ursodeoxycholic acid methyl ester, epideoxycholic acid, ursocholanic acid, epilithocholic acid, and all 3 cholic acid. Unless otherwise stated, the bile acids all derive from 5β-cholanic acid (i.e., the joint between rings A and B is in cis configuration). Since bile acids contain an ionized carboxylate in the C24 position at physiological pH (as in the experimental conditions disclosed herein), no distinction is made between the ionized (-ate suffix) and the nonionized forms (-olic acid suffix), e.g., lithocholic acid and lithocholate can be used indistinctly.

As used herein, “BK channels” refer to large conductance, calcium-activated and voltage-activated potassium channels, which allow potassium to leave the cytoplasm under physiological conditions when activated by membrane voltage and/or intracellular calcium, resulting in membrane repolarlization/hyperpolarization and, thus, a decrease in cell excitability.

The terms “compound” and “molecule” are herein used interchangeably.

As used herein, the term “derivative” is intended to mean a compound, molecule or agent derived or obtained from a parent substance (for example, lithocholate).

The term “effective amount” as used herein refers to any amount of active compound, molecule or agent that elicits the desired biological or medicinal response (e.g. blood flow in blood vessel) in a tissue, system, animal or human that is being sought by a researcher, veterinarian, medical doctor or other clinician. In some embodiments, the “effective amount” can refer to the amount of active compound, molecule or agent that is sufficient for targeting tissues that comprise BK channel β₁ subunits in a subject.

As used herein, the term “modulation” refers to a change in a biological variable, such as the activity of an ion channel or diameter of a blood vessel using the methods and compositions of the instant application. For example, modulation by an agent can cause an increase or a decrease in blood vessel diameter according to the methods of the presently disclosed subject matter.

The term “pharmaceutically acceptable” as used herein refers to a material that is not biologically or otherwise undesirable, i.e., the material can be incorporated into a pharmaceutical composition administered to a subject without causing any undesirable biological effects or interacting in a deleterious manner with any of the other components of the composition in which it is contained. When the term “pharmaceutically acceptable” is used to refer to a pharmaceutical carrier, it is implied that the carrier has met the required standards of toxicological and manufacturing testing or that it is included on the Inactive Ingredient Guide prepared by the U.S. Food and Drug administration. In some embodiments, “pharmaceutically acceptable” refers to a material that is pharmaceutically acceptable in humans.

The term “subject” as used herein refers to any invertebrate or vertebrate species. The methods disclosed herein are particularly useful in the treatment of warm-blooded vertebrates. Thus, the presently disclosed subject matter concerns mammals and birds. More particularly, provided is the treatment of mammals such as humans, as well as those mammals of importance due to being endangered (such as Siberian tigers), of economic importance (animals raised on farms for consumption by humans), and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the treatment of birds, including the treatment of those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, provided is the treatment of livestock, including, but not limited to, domesticated swine (pigs and hogs), ruminants, horses, poultry, and the like.

As used herein, “treatment” means any manner in which one or more of the symptoms of a disorder are ameliorated or otherwise beneficially altered. Thus, the terms “treating” or “treatment” of a disorder as used herein includes: reverting the disorder, i.e., causing regression of the disorder or its clinical symptoms wholly or partially; preventing the disorder, i.e. causing the clinical symptoms of the disorder not to develop in a subject that can be exposed to or predisposed to the disorder but does not yet experience or display symptoms of the disorder; inhibiting the disorder, i.e., arresting or reducing the development of the disorder or its clinical symptoms; attenuating the disorder, i.e., weakening or reducing the severity or duration of a disorder or its clinical symptoms; or relieving the disorder, i.e., causing regression of the disorder or its clinical symptoms. Further, amelioration of the symptoms of a particular disorder by administration of a particular composition refers to any lessening, whether permanent or temporary, lasting or transient that can be attributed to or associated with administration of the disclosed composition.

### II. REPRESENTATIVE EMBODIMENTS

The presently disclosed subject matter provides methods and compositions for modulating BK channel activity and vasodilation, for screening of compounds capable of modulating BK channel activity mediated through channel accessory β₁-subunits, and for methods of treatment. The present subject matter discloses that LC reversibly increases the diameter of pressurized resistance cerebral arteries by ˜10%, which would result in ˜30% increase in cerebral blood flow. In addition, the LC-induced vasodilation occurs via myocyte BK channels (see, by way of illustration, Example 2). LC activates BK channels in isolated myocytes through a destabilization of channel long-closed states without modifying unitary conductance (see, by way of illustration, Example 3). Channel accessory β₁-subunits, which are predominant in smooth muscle, are necessary for LC to modify channel activity (see, by way of illustration, Examples 3 and 5). In contrast, β₄-subunits, which are predominant in neuronal tissues, fail to evoke LC sensitivity (see, by way of illustration, Example 3). Accordingly, in some embodiments the presently disclosed subject matter provides LC and LC-like compounds that induce cerebrovascular dilation through molecular interaction with BK channel β₁-subunits.

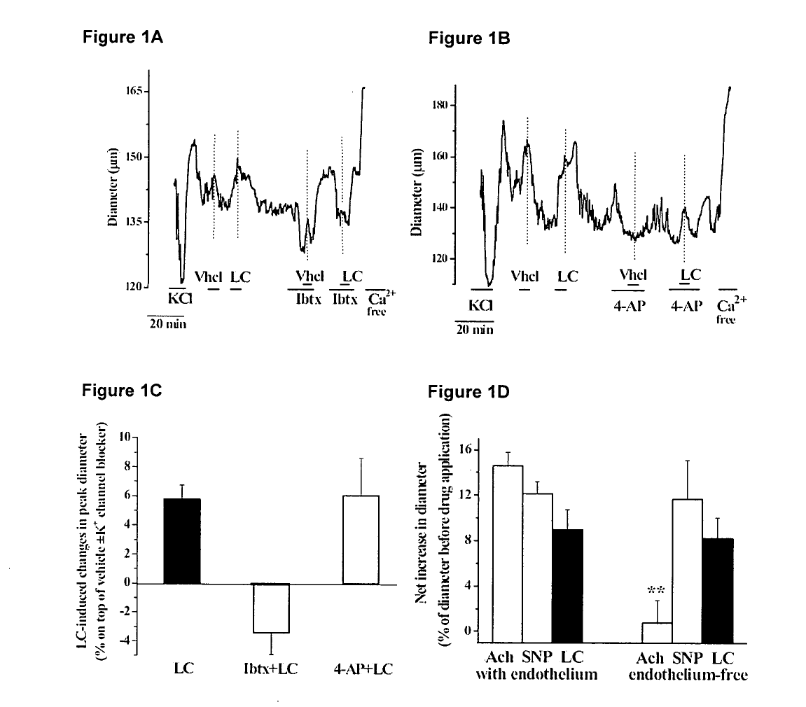

In some embodiments of the presently disclosed subject matter, the LC and LC-like compounds induce dilation of small cerebral arteries. In some embodiments, the modulation of blood vessel diameter can be used to calculate the corresponding modulation of cerebral blood flow. Particularly, changes in artery diameter are related to changes in cerebral blood flow by a factor of approximately 3. Such methods are described in Gourley, J. and Heistad, D. (1984) Am J Physiol 246: H52-H58, herein incorporated by reference in its entirety. Accordingly, in some embodiments, the LC and LC-like compounds of the presently disclosed subject matter induce dilation of small cerebral arteries by about a 10% increase in diameter, resulting in an increase in cerebral blood flow of about 30%. While endothelial-mediated vasodilation is impaired in several processes that affect cerebral vessels, such as atherosclerosis and vasospasm, dilation of small resistance arteries induced by the LC and LC-like compounds of the presently disclosed subject matter does not require a functional endothelium (see FIG. 1). Accordingly, the compositions of the presently disclosed subject matter comprising LC and LC-like compounds that induce dilation of small cerebral arteries can be clinically useful as cerebrovascular dilators.

Further, the LC and LC-like compounds of the presently disclosed subject matter lack the widespread hormonal actions of several other steroids. For example, while other steroids have been shown to activate Bk channels, including, 17β-estradiol (Valverde et al., 1999), xenoestrogens (Dick and Sanders, 2001; Perez, 2005), androgens (Deenadayalu et al., 2001) and glucocorticoids (King et al., 2006)), the effects of these agents on cerebrovascular myocyte BK channels and/or tone have not been demonstrated and these steroids have widespread hormonal actions which may preclude/limit their clinical use as vasodilators.

LC action on BK channels differs in several critical aspects from those of other steroids reported to modulate BK channels. For example, while 17β-estradiol increases BK (hslo) channel activity at micromolar (1-30 μM) concentrations by interacting with the channel β₁-subunit (Valverde et al., 1999), 17β-estradiol was also found to be a potent activator of BK channels containing either β2- or β₄-subunits (King et al., 2006). Furthermore, it has been reported that 17β-estradiol at submicromolar concentrations (0.01-1 μM) can modulate BK activity through an interaction between the steroid and the channel α-subunit (Korovkina et al., 2004). Finally, it has been suggested that 17β-estradiol dilation of coronary arteries via BK channels is not the result of a direct action on the channel but mediated through NO/cGMP-mediated pathways (White et al., 2002).

Tamoxifen (a xenoestrogen), and tamoxifen analogs, have been reported to have complex actions on BK activity, including, both an increase and decrease in Pₒ, which is reportedly related to basal Pₒ before drug application (Dick and Sanders, 2001; Perez, 2005; Duncan, 2005). In contrast, the presently disclosed subject matter shows an LC induced increase in Pₒ at all voltages, Ca²⁺ᵢ, and levels of Pₒ tested. Furthermore, under some conditions (Perez, 2005; Duncan, 2005) the β₁-subunit is not necessary for tamoxifen to evoke its complex actions on BK channels, the α-subunit being sufficient. Finally, tamoxifen and tamoxifen analogs decrease unitary current amplitude at concentrations as low as 1-10 μM (Duncan, 2005). This action might counterbalance the tamoxifen induced increase in Pₒ, with consequent reduction in drug potentiation of total BK current and, thus, vasodilation. In contrast, a β₁-subunit mediated increase in Pₒ and lack of effect on unitary conductance are observed at all LC concentrations. Thus, unlike tamoxifen and tamoxifen analogs, LC modulation of BK channel function appears to be limited to that of a gating modifier.

Cholesterol has been shown to reduce BK channel Pₒ at concentrations found in cell membranes; an activity that is opposite to that of LC induced increases in BK channel Pₒ. In addition, α-subunits are sufficient for cholesterol action on BK channel Pₒ (Bolotina et al., 1989; Crowley et al., 2003). Further, corticosterone has been shown to activate β₄-containing BK channels more effectively than P₂-containing BK channels, the opposite being true for dehydroepiandrosterone. Testosterone appears not to discriminate among channels containing these two β-subunits (King et al., 2006). In contrast, LC concentrations that are maximally effective in activating cbv1+β₁ channels do not modulate cbv1+β₄ channels.

Given the non-specific effects of most steroids on BK channels, the present showing that LC and LC-like compounds specifically affect BK channel activity through β₁ subunits was unexpected. Accordingly, the presently disclosed subject matter demonstrates that LC and LC-like compounds are uniquely useful tools for probing the presence of functional β₁-subunits and/or modulating smooth muscle BK channel activity. Furthermore, the presently disclosed revelation that LC and LC-like compounds can induce dilation of small resistance arteries demonstrates that these compounds can be clinically useful as cerebrovascular dilators. This discovery thereby provides methods and compositions for the important, but unmet need, for safer and more effective vasodilators.

Therefore, in some embodiments of the presently disclosed subject matter, methods are provided for screening for safer and more effective pharmacological agents for the treatment of cerebrovascular ischemic disease. In some embodiments, the specific interaction of LC with myocyte BK β₁-subunits that leads to cerebrovascular dilation is exploited by using methods of screening against the β₁-subunit. For example, in some embodiments methods of screening candidate compositions for an ability to modulate myocyte BK channel activity are provided, comprising, establishing a test sample comprising a myocyte BK channel β₁ subunit, measuring the interaction, effect, or combination thereof, of a candidate composition on the test sample to thereby determine the ability of the candidate composition to modulate myocyte BK channel activity. In some embodiments, the candidate composition comprises a lithocholate, or an analog or derivative thereof. In some embodiments, the myocyte is a small resistance artery myocyte.

In some embodiments of the presently disclosed subject matter, methods are provided for screening for candidate compositions useful in the treatment of a disorder where increasing myocyte BK channel activity can attenuate, revert or prevent the disorder, comprising, establishing a test sample comprising a myocyte BK channel β₁ subunit, and measuring the interaction, effect, or combination thereof, of a candidate composition on the test sample to thereby determine the ability of the candidate composition to modulate myocyte BK channel activity and to attenuate, revert or prevent the disorder. In some embodiments, the candidate composition comprises a lithocholate, or an analog or derivative thereof. In some embodiments, the myocyte is a small resistance artery myocyte. In some embodiments, the attenuation, reversion or prevention of the disorder is mediated at least in part through modulation of blood pressure and/or blood flow. In some embodiments, the attenuation, reversion or prevention of the disorder is mediated at least in part through vasodilation. In some embodiments, the vasodilation is characterized by an increase in blood vessel diameter of about 10 percent.

In some embodiments of the presently disclosed subject matter, methods are provided for treating a subject having a disorder, the methods comprising, administering to the subject a composition comprising a compound capable of increasing myocyte BK channel activity mediated by the BK channel β₁ subunit, wherein increasing myocyte BK channel activity can attenuate, revert or prevent the disorder. In some embodiments, the compound comprises a lithocholate, or an analog or derivative thereof. In some embodiments, the administration of the composition comprising the compound capable of increasing myocyte BK channel activity results in decreases in blood pressure and/or increases in blood flow. In some embodiments, the increase in blood flow is mediated at least in part through vasodilation. In some embodiments, the vasodilation occurs at least in part in small resistance arteries. In some embodiments, the small resistance arteries are small cerebral arteries. In some embodiments, the vasodilation in the small cerebral arteries is about a 10 percent increase in arterial diameter. In some embodiments, the increase in blood flow in the small cerebral arteries is about 30 percent. In some embodiments, the subject is a mammal.

In some embodiments of the presently disclosed subject matter, pharmaceutical compositions are provided that are useful for increasing myocyte BK channel activity in a subject, wherein the composition comprises an effective amount of a lithocholate or an analog or derivative thereof, and a pharmaceutically acceptable carrier.

In some embodiments of the presently disclosed subject matter, pharmaceutical compositions are provided that are useful for increasing myocyte BK channel activity in a subject, wherein the BK channel activity is mediated by the BK channel β₁ subunit.

In some embodiments, the LC and LC-like compounds of the presently disclosed subject matter are useful for selectively targeting tissues and organs that contain high amounts of β₁-subunits. LC and LC structural analogs appear not to interact with α+β4 channel complexes and LC and LC structural analog modulation of BK channels differs from that of other steroid-based molecules in a number of ways. For example, dehydrosoyasaponin-1 (DHS-1), a complex molecule that contains a steroidal nucleus, has been reported to modulate BK channels through an interaction with the β₁-subunit (Giangiacomo et al., 1998), but DHS-1 is effective only when accessing the channel from the cytosolic side of the membrane. In contrast, LC and LC structural analogs are similarly effective when applied to the external or internal membrane surface. This limits the application of DHS-1 to tissue and organ studies. In addition, DHS-1 action is strongly voltage-dependent, while LC action is not. It has not been reported whether BK channel subunits other than β₁ can render BK channels sensitive to nanomolar concentrations of DHS-1. Therefore, in some embodiments, the LC and LC-like compounds of the presently disclosed subject matter can be used to selectively target tissues and organs that contain high amounts of P₁-subunits (i.e., smooth muscle), as opposed to others rich in α+β₄ complexes (i.e., CNS, in which BK channel activation would affect neuronal excitability (Meredith et al., 2006) and/or neurotransmitter release (Brenner et al., 2005)).

In some embodiments of the presently disclosed subject matter, methods are provided for selectively targeting tissues comprising BK channel β₁ subunits in a subject, comprising, administering a composition comprising an effective amount of a lithocholate or an analog or derivative thereof, to the subject, whereby tissues comprising BK channel β₁ subunits are targeted. In some embodiments, the tissues targeted are smooth muscle tissues. In some embodiments, the composition of the presently disclosed subject matter comprises one or more imaging agents. In some embodiments, the imaging agent is an x-ray agent and can include, for example, barium sulfate, ioxaglate meglumine, ioxaglate sodium, diatrizoate meglumine, diatrizoate sodium, ioversol, iothalamate meglumine, iothalamate sodium, iodixanol, iohexyl, iopentol, iomeprol, iopamidol, iotroxate meglumine, iopromide, iotrolan, sodium amidotrizoate, meglumine amidotrizoate, and the like. In some embodiments, the imaging agent is a MRI agent and can include, for example, gadopentetate dimeglumine, ferucarbotran, gadoxetic acid disodium, gadobutrol, gadoteridol, gadobenate dimeglumine, ferumoxsil, gadoversetamide, gadolinium complexes, gadodiamide, mangafodipir, and the like. In some embodiments, the imaging agent is an ultrasound agent and can include, for example, galactose, palmitic acid, SF₆, and the like. In some embodiments, the imaging agent is a nuclear agent and can include, for example, technetium (Tc^{99m}) tetrofosmin, ioflupane, technetium depreotide, technetium exametazime, fluorodeoxyglucose (FDG), samarium (Sm¹⁵³) lexidronam, technetium mebrofenin, sodium iodide (I¹²⁵ and I¹³¹), technetium medronate, technetium tetrofosmin, technetium fanolesomab, technetium mertiatide, technetium oxidronate, technetium pentetate, technetium gluceptate, technetium albumin, technetium pyrophosphate, thallous (TI²⁰¹) chloride, sodium chromate (Cr⁵¹), gallium (Ga⁶⁷) citrate, indium (In¹¹¹) pentetreotide, iodinated (I¹²⁵) albumin, chromic phosphate (P³²), sodium phosphate (P³²), and the like. According to a further embodiment, the imaging agent can include a combination of the above listed agents. In some embodiments, the composition further comprises a pharmaceutically acceptable carrier. In some embodiments, the subject is a mammal.

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Materials and Methods

Artery Diameter Measurement. Middle cerebral arteries were isolated from adult male Sprague-Dawley rats (=250 g) or 8- to 12-week-old β₁-knockout and C57BL/6 control mice. Rats and mice were decapitated using a guillotine and sharp scissors, respectively. These procedures were approved by the Institutional Animal Care and Use Committee from The University of Tennessee Health Science Center, an AMLAC-accredited institution. Pressurization of arteries was performed as described (Liu et al., 2004). Endothelium was removed by passing an air bubble into the vessel lumen for 90 sec. Diameter changes were monitored through an inverted microscope (Nikon Eclipse TS100, Nikon Corp., Tokyo, Japan), recorded on camera (Sanyo VCB-3512T, Sanyo Electric Corp., Japan), and transferred to a computer. Diameter data were acquired and analyzed using IonWizard 4.4 software (IonOptics Corp., Milton, Mass., United States of America).

Pressurized arteries were extraluminally perfused with physiological saline solution (PSS) (Liu et al., 2004) at a constant rate of 3.75 ml/min using a peristaltic pump Dynamax RP-1 (Rainin Instrument, Inc., Oakland, Calif., United States of America). At this rate, complete washout of the iberiotoxin (Ibtx) effect required >45 min. To keep basal tone under steady behavior, we shortened this period by increasing flow rate ˜3 times during washout of Ibtx, which sometimes evoked a flow-induced dilation (FIG. 1A). Equal volumes (25 ml) of vehicle- vs. LC-containing solutions were applied at equal, constant rate (see above) to the pressurized arterial segment in the chamber via a gravity system. Drugs were dissolved to make stock solutions (see Chemicals) and diluted in PSS to final concentration.

Myocyte Isolation. Basilar and middle cerebral arteries were dissected out from each brain under a stereozoom microscope (Nikon C-PS, Tokio, Japan) and placed into ice-cold “dissociation medium” (DM) (mM): 0.16 CaCl₂, 0.49 EDTA, 10 HEPES, 5 KCl, 0.5 KH₂PO₄, 2 MgCl₂, 110 NaCl, 0.5 NaH₂PO₄, 10 NaHCO₃, 0.02 phenol red, 10 taurine, 10 glucose. Each artery was cut into 1-2 mm long rings (˜30 rings/experiment). Rings were put in 3 ml DM containing 0.03% papain, 0.05% bovine serum albumin (BSA) and 0.004% dithiothreitol (DTT) for 15 min at 37° C. in a polypropylene centrifuge tube, and then incubated in a shaking water bath at 37° C. and 60 oscillations/min. for 15 min. The prepartion was then centrifuged several times as described (Liu et al., 2004). After the final centrifugation, the supernatant was discarded, and the pellet resuspended in 3 ml of DM containing 0.06% soybean trypsin inhibitor. Finally, the tissue was pipetted using a series of borosilicate Pasteur pipettes having fire-polished, diminishing internal diameter tips. The procedure rendered a cell suspension containing relaxed, individual myocytes (≧5 myocytes/field using a 40× objective) that could be easily identified under microscope (Olympus IX-70; Olympus America, Woodbury, N.Y., United States of America). The cell suspension was stored in ice-cold DM containing 0.06% BSA, and the cells were used for patch-clamping up to 4 h after isolation.

cRNA Preparation and Injection into Xenopus Oocytes. Full-length cDNA coding for cbv1-subunits was cloned from rat cerebral artery myocytes by PCR and ligated to the PCR-XL-TOPO cloning vector (Invitrogen Corp., Carlsbad, Calif., United States of America) (Jaggar et al., 2005). cDNA coding for cbv1-subunits was cleaved from the cloning vector by BamHI (Invitrogen Corp., Carlsbad, Calif., United States of America) and XhoI (Promega, Madison, Wis., United States of America) and directly inserted into the pOX vector for expression in Xenopus oocytes. pOX-cbv1 was linearized with NotI (Promega, Madison, Wis., United States of America) and transcribed in vitro using T3 polymerase. Beta,-subunit cDNA inserted into the EcoR I/Sal I sites of the pCl-neo expression vector was linearized with NotI and transcribed in vitro using T7 polymerase. Beta₄-subunit cDNA inserted into the pOx vector was linearized by NotI and transcribed using T3 polymerase. The mMessage-mMachine kit (Ambion Inc., Austin, Tex., United States of America) was used for transcription. The pOX vector and the cDNA coding for β₁-subunits were generous gifts from Aguan Wei (Washington University, Saint Louis, Mo., United States of America) and Maria Garcia (Merck Research Laboratories, Whitehouse Station, N.J., United States of America).

Oocytes were removed from Xenopus laevis and prepared as described (Dopico et al., 1998). cRNA was dissolved in diethyl polycarbonate-treated water at 5 (cbv1) and 15 (β₁ or β₄) ng/μl; 1-μl aliquots were stored at −70° C. Cbv1 cRNA was injected alone (2.5 ng/μp) or coinjected with either β₁ or β₄ (7.5 ng/μl) cRNAs, giving molar ratios ≧6:1 (β:α). cRNA injection (23 nl/oocyte) was conducted using a modified micropipette (Drummond Scientific Co., Broomall, Pa., United States of America). The interval between injection and patch-clamp recordings was 48-72 h.

Electrophysiology. Oocytes were prepared for patch-clamp recordings as described (Dopico et al., 1998). Single-channel and macroscopic currents were recorded from inside-out (I/O) or outside-out (O/O) patches. For experiments with oocytes, both bath and electrode solutions contained (mM) 135 K⁺ gluconate, 5 EGTA, 1 MgCl₂, 15 HEPES, 10 glucose, pH 7.35. For experiments with myocytes, KCl substituted for K⁺ gluconate. In all experiments, free Ca²⁺ in solution was adjusted to the desired value by adding CaCl₂. In most studies, free Ca²⁺ in electrode solution=10 μM. In O/O studies with 17β-estradiol, however, free Ca²⁺ in the electrode solution=0.3 μM. Nominal free Ca²⁺ was calculated with MaxChelator Sliders (C. Patton, Stanford University, California, United States of America) and validated experimentally using Ca²⁺-selective electrodes (Corning Incorporated Science Products Division, Corning, N.Y., United States of America).

Patch-recording electrodes were made as described (Dopico et al., 1998). Immediately before recording, the tip of each electrode was fire-polished on a microforge WPI MF-200 (World Precision Instruments, Inc., Sarasota, Fla., United States of America) to give resistances of 5-9 MD when filled with solution. An agar bridge with gluconate or Cl⁻ as the main anion (for oocyte and myocyte experiments, respectively) was used as ground electrode. After excision from the cell, the membrane patch was exposed to a stream of bath solution containing each agent at final concentration. Solutions were applied onto the patches using a pressurized system DAD12 (ALA Scientific Instruments, New York, N.Y., United States of America) via a micropipette tip with an internal diameter of 100 μm. Experiments were carried out at room temperature (21° C.).

Currents were recorded using an EPC8 amplifier (HEKA Electronics, Lambrecht/Pfalz, Germany) at 1 kHz using a low-pass, eight-pole Bessel filter 902LPF (Frequency Devices, Haverhill, Mass., United States of America). Data were digitized at 5 kHz using a Digidata 1320A A/D converter and pCLAMP 8.0 (Molecular Devices, Union City, Calif., United States of America). For macropatch recordings, G/Gmax-V data were fitted to the Boltzmann function:

G(V)=Gₘₐₓ/(1+e^{(-V+V}^{_{0.5)}}/^{k})

Using the slope (k) of the G/Gₘₐₓ vs. V plots, z (i.e., 1/k) was calculated as 1/k=RT/F, where R, T, and F have their usual meaning. As an index of channel steady-state activity, the product was used of the number of channels in the patch (N) and the channel open probability (Pₒ). NPₒ was obtained from all-points amplitude histograms from ≧30 sec of continuous recording under each experimental condition.

Chemicals. All chemicals were purchased from Sigma (St. Louis, Mo., United States of America), with the exception of 5β-cholanic acid 3α-ol (LC) (Steraloids, Inc., Newport, R.I., United States of America) and Ibtx (Alomone Labs Ltd., Jerusalem, Israel). On the day of the experiment, an LC stock solution (333 mM) in dimethyl sulfoxide (DMSO) was freshly prepared by sonication for 5 min. For arterial tone experiments, the LC stock was diluted 1/10 in DMSO and further diluted in PSS to render final LC concentration. Solution containing vehicle (0.1% DMSO; V/V) was used as control perfusion. For electrophysiological recordings, the LC stock solution was diluted 1/10 in 95% ethanol and further diluted with bath solution to render final LC concentration (3-1,000 μM). The DMSO/ethanol vehicle (≦0.1/≦0.86% final concentrations) in bath solution was used as control.

Data Analysis. Artery diameter response to each compound is shown as a percentage of the diameter obtained before compound application. Arterial diameter and electrophysiological data were analyzed with IonWizard 4.4 (IonOptics Corp., Milton, Mass., United States of America) and pCLAMP 8.0 (Molecular Devices, Union City, Calif., United States of America). Further analysis, plotting, and fitting were conducted using Origin 7.0 (Originlab Corp., Northampton, Mass., United States of America) and InStat 3.0 (GraphPad Software Inc., San Diego, Calif., United States of America).

Statistical analysis was conducted using either one-way ANO/A and Bonferroni's multiple comparison test or paired Student's t-test; significance was set at p<0.05. Data are expressed as mean ±SEM; n=number of patches/arteries.

### Example 2

### Lithocholate Dilates Small, Resistance Arteries via BK Channels

After myogenic tone development at 60 mm Hg, intact arteries reached a diameter of 154.4±5.2 μm (n=17). Maximal contraction and dilation were checked by perfusing the vessel with 60 mM KCl at the beginning, and Ca²⁺-free solution at the end of each experiment (FIGS. 1A & 1B). In all cases (n=17), application of 45 μM LC, that is, concentrations well below LC's critical micellar concentration (CMC) (≧1 mM under our recording conditions) (Roda et al., 1995) caused a significant increase in peak arterial diameter: +6% on top of a transient increase in diameter caused by vehicle-containing solution (FIGS. 1A-1C). LC-induced dilation was not only larger than that caused by vehicle but also more sustained (˜2.8 times longer); for example, by the time the vehicle effect had totally vanished, LC-induced dilation still represented 109.9±1.7% of the initial arterial diameter determined before any compound application (n=17; p<0.01). The differential vasodilation caused by LC vs. vehicle is most evident from the area under the curve values (integrals) corresponding to the change in diameter as a function of time: 18,369±3,964 vs. 10,262±2,574 (p<0.01) (Table 1). Notably, the net increase in cerebral artery diameter caused by LC over pre-LC values (+9.9%) is expected to cause a marked increase (˜30%) in cerebral blood flow (CBF), because changes in artery diameter are related to changes in CBF by a factor of ˜3 (Gourley and Heistad, 1984).

TABLE 1
Characteristics of LC-induced vs. vehicle-induced vasodilation.

| | Vehicle | Lithocholic |
|---|---|---|
| Parameter | (0.1% DMSO) | acid (45 μM) |
| Rise time (sec) | 244.2 ± 16.1 | 231.9 ± 21.9 |
| Maximal effect (% from diameter | 110.8 ± 1.6 | 116.58 ± 1.9** |
| before compound application) | | |
| Time for full recovery (sec) | 347.4 ± 47.1 | 981.4 ± 129.8** |
| Integral (area under the curve) | 10,262 ± 2,574 | 18,369 ± 3,964** |
| from the time of drug application | | |
| until complete washout of effect | | |
| Effect remaining after ~6 min of | None | 109.9 ± 1.7** |
| washout (percentual increase | | |
| from diameter before agent | | |
| application) | | |

**Significantly different from control) vehicle-containing solution (p < 0.01) (paired Student's t-test).

Rat cerebral artery diameter is critically controlled by myocyte BK channel activity (Jaggar et al., 2000). Since these channels are selectively blocked by nanomolar concentrations of Ibtx (Liu et al., 2004), this peptide was used to determine any possible contribution of BK channels to LC-dilation. As expected, bath application of 55 nM Ibtx caused a robust decrease in the diameter of intact arteries (−11.8±3.4%) (n=4) (FIG. 1A). Remarkably, LC dilation was completely lost when the steroid was applied in addition to Ibtx (FIGS. 1A & 1C). In the presence of Ibtx, LC caused some reduction in diameter (−3.4%), which could be related to the well-known increase in cytosolic Ca²⁺ caused by bile acids (Thibault and Ballet, 1993). These data indicate that LC fails to dilate small, resistance arteries when BK channels are specifically blocked.

Cerebrovascular smooth muscle tone is also controlled by voltage-gated K⁺ channels (K_{V}) other than BK (Faraci and Sobey, 1998). To determine the selectivity of BK channel involvement in LC dilation, LC action was evaluated in the presence of 4-aminopyridine (4-AP) which, at sub- to low millimolar concentrations, blocks most K_{V} but not BK channels in rat cerebral arteries (Liu et al., 2004). Applying 0.8 mM 4-AP caused an immediate decrease in diameter (−9.7±2.3%, n=4) (FIG. 1B). In contrast to the Ibtx results, the change in peak diameter caused by LC in the presence of 4-AP was identical to that determined in the absence of K_{V} blocker (+6% over pre-LC values; FIG. 1C). While some contribution of K_{V} channels other than BK to LC dilation cannot be ruled out, these results indicate that K_{V} channels other than BK do not play a major role in LC dilation of pressurized small, resistance cerebral arteries. Furthermore, 45 μM LC on top of 4-AP almost totally reverted the vasoconstriction caused by the Kv channel blocker (FIG. 1B), underscoring the effectiveness of BK channel-targeting by LC in reversing cerebrovascular constriction driven by voltage-dependent mechanisms. In contrast to LC dilation, the small and transient increase in diameter caused by vehicle was unmodified by Ibtx (FIG. 1A) but somewhat decreased by 4-AP (FIG. 1B). The differences in time-course and magnitude (Table 1), together with their differential modulation by selective channel blockers, indicate that LC and vehicle dilation of cerebral arteries are mediated by different ionic mechanisms, the former via BK channels.

Finally, to rule out that endothelial factor(s) could be mediating or, at least, modulating LC-induced dilation, LC action was studied in de-endothelized arteries and compared to that in intact vessels. LC-induced dilation is similar in intact vs. endothelium-denuded arteries (n=5). The presence of a functional endothelium was assessed by vascular responses to endothelium-dependent (acetylcholine; 10 μM) and independent (sodium nitroprusside; 10 μM) vasodilators. Indeed, while vasodilation in response to acetylcholine was lost (n=4), sodium nitroprusside-induced dilation was fully preserved in de-endothelized arteries (n=5) (FIG. 1D). Notably, LC increase in diameter of de-endothelized arteries was not significantly different from that of intact arteries (FIG. 1D). Thus, LC-induced dilation of small, resistance cerebral arteries is independent of a functional endothelium. Collectively, data shown in FIGS. 1A-1D suggest that LC dilation of small cerebral arteries is due to LC targeting of myocyte BK channels.

In summary, it has been demonstrated that LC (45 μM) reversibly increases the diameter of pressurized resistance cerebral arteries by ˜10%, which would result in ˜30% increase in cerebral blood flow. LC action is independent of endothelial integrity, prevented by 55 nM iberiotoxin, and unmodified by 0.8 mM 4-aminopyridine, indicating that LC causes vasodilation via myocyte BK channels.

### Example 3

### Lithocholate Directly Activates Myocyte BK Channels via the Channel β₁ Subunit

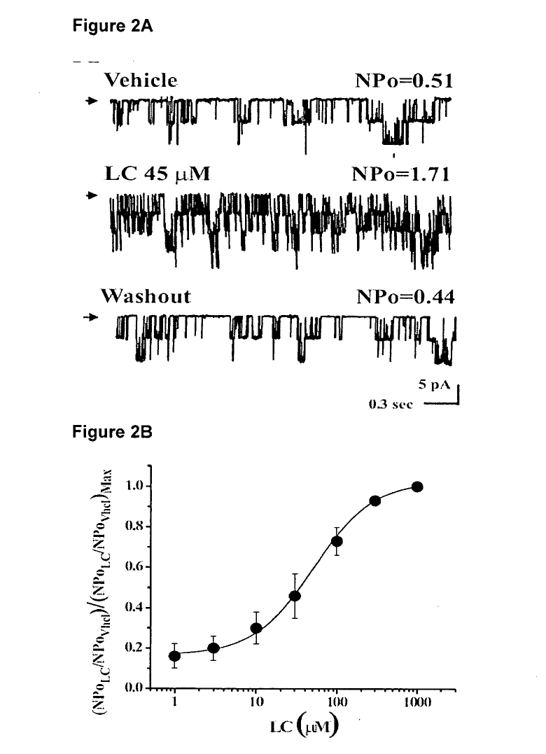

To determine whether LC directly targets BK channels in cerebral artery myocytes, drug action on channel activity was studied by using I/O patches with the membrane potential and free Ca²⁺ᵢ set at values (−40 to −30 mV and 3 μM) similar to those obtained in cerebrovascular myocytes during contraction (Knot and Nelson, 1998; Perez et al., 2001). After excision, the patch was exposed to vehicle-containing solution, and BK NPₒ was recorded for no less than 1 minute. Then, applying LC-containing (1-1,000 μM) solution reversibly increased NPₒ in a concentration-dependent fashion: EC₅₀=46±6 μM, Eₘₐₓ˜300 μM (FIGS. 2A & 2B). At Eₘₐₓ, NPₒ reached 350% of control, this ceiling remaining steady up to 1 mM LC. Concentrations above 1 mM (i.e., close to the CMC for LC under the recording conditions used) (Roda et al., 1995) systematically resulted in loss of gigaseals, likely due to a micelle-mediated detergent effect. Thus, LC maximally increases BK channel activity at aqueous concentrations in which LC monomers predominate, as opposed to a detergent action on the membrane due to micelle formation in solution. LC increase in NPₒ was observed in membrane patches that were excised from the myocyte >5 min before applying LC under continuous bath perfusion in the absence of nucleotides. Therefore, LC action does not require cell integrity or the continuous presence of cytosolic messengers. Rather, it is due to a direct interaction between the steroid and the BK channel complex itself and/or its immediate proteolipid environment.

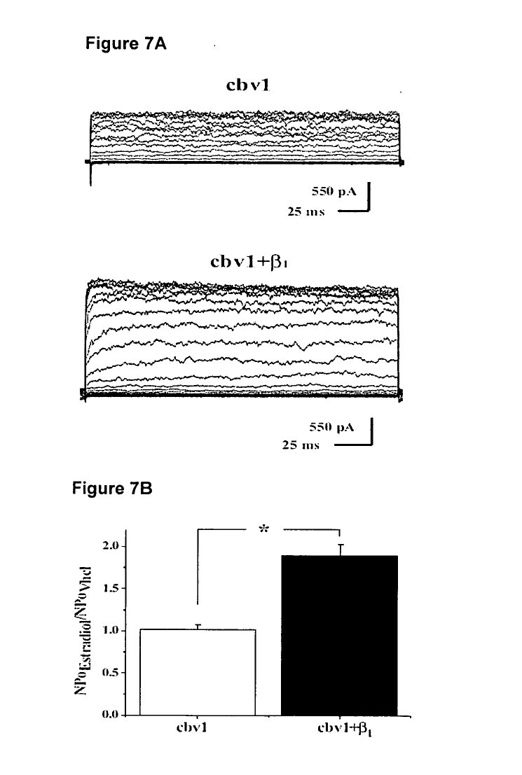

To determine which subunit of the channel complex is involved in sensing LC with eventual increase in NPo, electrophysiological recordings were performed in I/O patches from Xenopus oocytes expressing either homomeric cbv1 or heteromeric cbv1+β₁ channels under identical conditions. To evoke measurable levels of Pₒ within a sec-min time frame in the absence of β₁-subunits, these studies were conducted at Ca²⁺ᵢ=10 μM, at either positive or negative Vm (+20 or −20 mV). Considering that LC effect on BK channel NPₒ is voltage-independent, data obtained at both voltages were pooled. Cbvl-subunits expressed in oocytes rendered macro- and microscopic currents that showed all major biophysical and pharmacological features of BK currents (Jaggar et al., 2005). The presence of functional β₁-subunits was confirmed by macroscopic currents characteristics (Brenner et al., 2000a) (slower activation kinetics, increased apparent Ca²⁺-sensitivity with a shift in V_{0.5} of ˜20 mV towards negative potentials) and channel activation by bath application of 10 μM 17β-estradiol to the extracellular surface of O/O patches (Valverde et al., 1999) (FIGS. 7A & 7B; FIG. 2B).

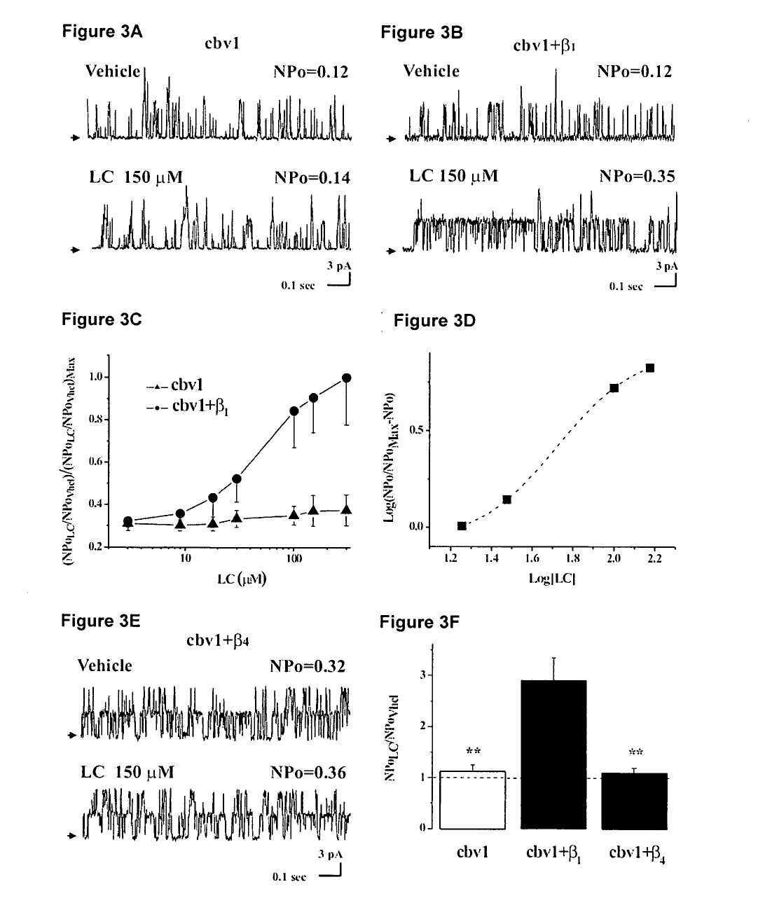

In contrast to the results obtained with native BK channels in cerebrovascular myocytes, application of LC as high as 150 μM (on top of vehicle) to the internal side of I/O patches failed to activate homomeric cbv1 channels, with average NPₒ reaching 112±13% of control (p>0.05; n=4) (FIGS. 3A & 3C). Thus, LC activation of cerebrovascular BK channels requires the presence of β₁-subunits and/or some other component of the myocyte membrane that is missing in the heterologous expression system. As found with native BK channels, however, LC (3-300 μM) caused a reversible and concentration-dependent increase in NPₒ of heteromeric cbv1+β₁ channels (FIGS. 3B & 3C), with EC₅₀=43.5±4.7 μM and Eₘₐₓ˜300 μM, at which NPₒ reached 290±45% of control. These values are practically identical to those of native BK channels (see above), indicating that differences in composition/organization between rat cerebrovascular myocyte and Xenopus oocyte membranes are not critical in LC action on BK channels. The identical LC responses of native cerebrovascular BK and cbv1+β₁ channels appear to indicate the involvement of a common target(s) mediating LC action in these two systems, possibly the β₁-subunit itself.

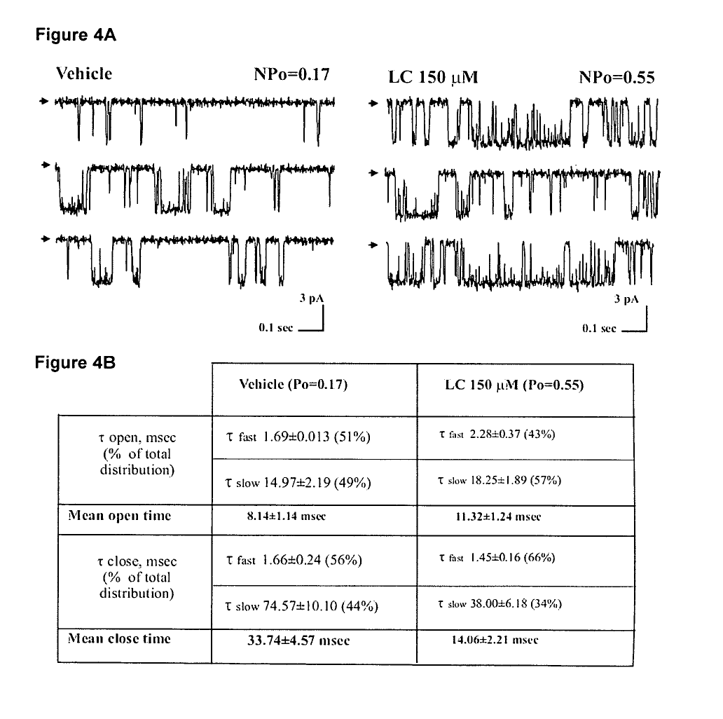

A Hill-like plot for LC-activation of cbv1+β₁ channels renders a slope (apparent Hill coefficient) of 1.3 (FIG. 3D), which suggests the involvement of at least two “sites” in the cbv1+β₁ complex for LC to increase NPo. An increase in the number of channels (N) might contribute to the overall increase in NPₒ caused by LC. Data from patches where N=1 (FIG. 4A), however, show an increase in PO that is similar to the increase in NPₒ in patches containing an unknown N. Thus, LC action on BK steady-state activity appears to be solely determined by an increase in Pₒ. Given the apparent Hill coefficient of 1.3, the increase in Pₒ appears to require the interaction of at least two LC molecules with the P₁-subunits of the channel complex.

From the channel dwell-time distributions in patches (n=2) where N=1 (FIG. 4A), both open and mean closed times were calculated (Dopico et al., 1998). Both distributions could be well-fitted with double exponential functions, indicating the existence of at least two open and two closed states. Lithocholate increased the channel mean open time, which reached 137% of control. This enhancement resulted from an LC-induced increase in the average duration of both short and long open channel events, with an accompanying mild shift in the open channel distribution towards longer openings; the long open state(s) accounted for 49% and 57% of total open events in vehicle and LC, respectively (FIGS. 4A & 4B). In addition, LC drastically decreased the channel mean closed time, which reached 41% of control. This reduction was primarily caused by a robust reduction in the average duration of channel long closed events, and also a shift towards briefer closures; the long close state(s) accounted for 44% and 34% of total close events in vehicle and LC, respectively (FIGS. 4A & 4B).

In summary, the LC-induced increase in channel Pₒ results primarily from destabilization of channel long closed states, eventually reducing by more than half the channel mean closed time. These changes in channel kinetics with consequent increase in Pₒ occurred in the absence of significant change in unitary conductance: 228.6±3.7 vs. 234.0±4.6 pS in symmetric 135 mM K⁺ (n=4; NS). Thus, LC modification of BK channel function is limited to modification of channel gating.

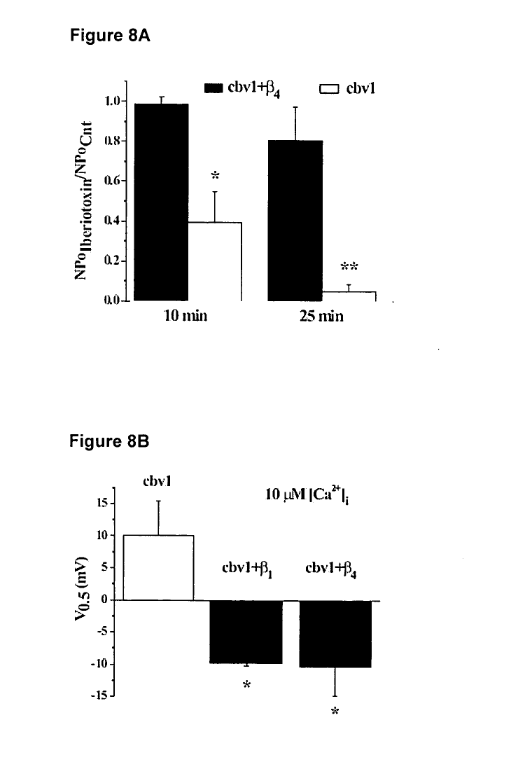

To determine whether LC increase in BK Pₒ is selectively mediated by the β-subunit type (β₁) that is predominant in smooth muscle or could be mediated by other channel accessory-subunits, LC action on cbv1+β₄ channels was tested. When coexpressed with α-subunits, β-subunits introduce a hyperpolarizing shift in V_{0.5} similar to that caused by α+β₁ coexpression. In addition, β₄-subunits render the BK complex relatively resistant to Ibtx (Meera et al., 2000). Confirmation of this phenotype is demonstrated by the data in FIGS. 8A & 8B. Under conditions identical to those used with cbv1 and cbv1+β₁ channels, cbv1+β₄ channels were consistently refractory to LC action (8/8 patches), even when tested at concentrations (150 μM) that were close to Eₘₐₓ in both cbv1+β₁ and native BK channels (FIGS. 3E & 3F); cbv1+β₄ NPₒ reached 109±11% of control (NS, also not significantly different from LC action on cbv1 homomeric channels). Therefore, β₁- but not β-subunits confer LC sensitivity to cerebrovascular BK channels.

In summary, both vasodilation and full channel activation occur at LC concentrations well below the steroid CMC, which indicates that these actions are due to the presence of LC monomers in the aqueous phase and not to nonspecific detergent effects on the membrane caused by LC micelles in solution. Some bile acid analogs that are effective “detergents” (positive curvature-forming lipids) fail to activate myocyte BK channels (Dopico et al., 2002). In addition, LC monomers activate the channel independently of cell integrity, cytosolic mediators, or steroid metabolism. Collectively, these results strongly support that LC activates BK channels via a selective interaction with a steroid target secondary to the presence of LC monomers in solution. Further, LC activates BK channels in isolated myocytes through a destabilization of channel long-closed states without modifying unitary conductance. Channel accessory P₁-subunits, which are predominant in smooth muscle, are necessary for LC to modify channel activity. In contrast, β₄-subunits, which are predominant in neuronal tissues, fail to evoke LC sensitivity. LC activation of cbv1+β₁ and native BK channels display identical characteristics, including EC₅₀ (46 μM) and Eₘₐₓ (=300 μM), strongly suggesting that the cbv1+β₁ complex is necessary and sufficient to evoke LC action.

### Example 4

### Lithocholate Effectively Activates BK Channels within Physiological Ranges of Ca²⁺ᵢ and Membrane Voltage

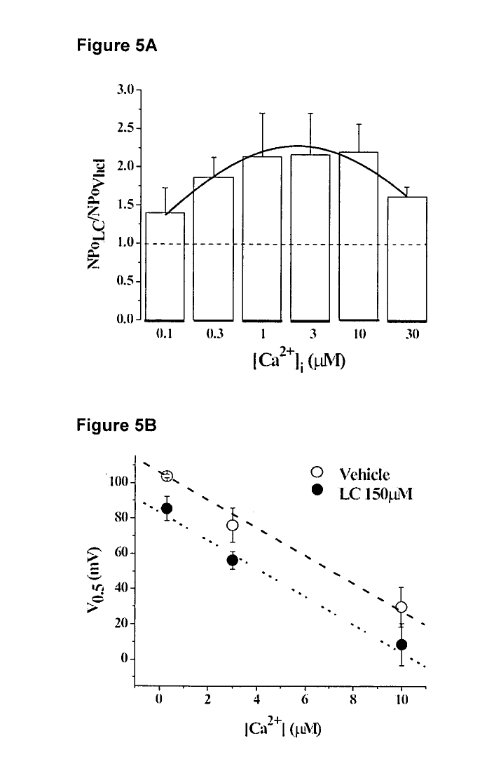

β₁-subunits modulate both Ca²⁺-dependent and -independent channel gating, resulting in an increase in the apparent Ca²⁺ sensitivity of the channel. This effect is more pronounced at Ca²⁺ᵢ that effectively increases Pₒ (Meera et al., 1996; Nimigean and Magleby, 2000). On the other hand, the lateral chain of LC contains a carboxyl that is ionized at physiological pH, raising the possibility that LC action could be modified by transmembrane voltage. Thus, the Ca²⁺ᵢ and voltage-dependence of LC action on cbv1+β₁ channel Pₒ was explored by using a wide voltage range (±150 mV) and Ca²⁺ᵢ levels that expanded those in the myocyte under physiological conditions (0.15-0.3 μM at rest; up to 10-30 μM in the vicinity of BK channels during contraction) (Perez et al., 2001; Liu et al., 2004). Even at non-physiological, very positive voltages (+80 mV), LC potentiation of BK NPₒ was unnoticeable when recorded in solutions having zero Ca²⁺ added plus 10 mM EGTA to chelate trace amounts of the divalent ion (n=3). This is consistent with LC modulating channel gating via a β₁-mediated mechanism, because at “zero” or 2+subactivatory Ca²⁺ᵢ, β₁-subunit modification of gating does not translate into an evident change in overall Pₒ (Nimigean and Magleby, 2000). Furthermore, LC-activation (as a percentage of NPₒ in vehicle) increased with Ca²⁺ᵢ: from 139.9±32.9 (n=4; p<0.05) at 0.1 μM Ca²⁺ to a maximal effect of 244.1±58.9% (n=3; p<0.01) at 1 μM Ca²⁺. This maximum remained steady within the 1 to 10 μM Ca²⁺ᵢ range (n=16), and decreased with higher Ca²⁺ᵢ (e.g., at 30 μM, NPₒ in LC reached 172.5±9.5% of control; p<0.05, n=3) (FIG. 5A). These data demonstrate that LC activates BK channels within a Ca²⁺ range that spans from resting levels to those reached during myocyte contraction. Remarkably, LC activation is most effective at Ca²⁺ levels reached near the BK channel during cerebral artery myocyte contraction (Perez et al., 2001).

Next, LC action on cbv1+β₁-mediated currents as a function of applied voltage was evaluated, exposing I/O macropatches to Ca²⁺ᵢ at which LC activation of BK channels is robust: 0.3, 3, and 10 μM Ca²⁺ᵢ. From G/Gₘₐₓ vs. Vm plots fitted to a Boltzmann relationship, V_{0.5}=101.9±1.2 (n=3), 74±10 (n=3), and 32.3±11.2 mV (n=5), respectively, were obtained. At every Ca²⁺ᵢ: tested, LC (150 μM) shifted the V_{0.5} by ≈−17.7 mV (FIG. 5B) without changing the slope of the plot. Thus, at any constant Ca²⁺ᵢ, the effective valence (z) (i.e., an index of the minimum number of elementary charges that cross the electric field to gate the channel) was similar in the absence or presence of LC (e.g., at 10 μM free Ca²⁺ᵢ: z=1.24±0.29 vs. 1.26±0.2). These data suggest that LC does not interfere with the voltage-sensing process of channel gating. The lack of LC effect on z is also consistent with a α-mediated action on channel gating (Brenner et al., 2000a). Together, the data show that LC is an effective activator of BK channels via their β₁-subunits at physiologically relevant Ca²⁺ᵢ and voltages.

In summary, LC channel activation occurs within a wide voltage range and at Ca²⁺ concentrations reached in the myocyte whether at rest or during contraction.

### Example 5

### Lithocholate Fails to Induce Cerebrovascular Dilation in β₁-Knockout Mice

The data disclosed herein demonstrate that acute application of LC readily and reversibly increases the activity of native BK channels freshly isolated from small resistance arteries. Vascular smooth muscle BK channels are made of channel-forming α subunits (KCNMA1) and regulatory β₁ subunits (KCNMB1) (Orio et al., 2002). In contrast, BK α+β₄ subunits (KCNMB4) are predominant in neuronal tissues (Brenner et al., 2000a; Meera et al., 2000). After cloning α-subunits (termed “cbv1”; AY330293) from myocytes freshly isolated from rat resistance cerebral arteries, recombinant BK channels were used to demonstrate that the channel β₁-subunit acts as the LC sensor. In contrast, β₄-subunits fail to render BK channels sensitive to LC.

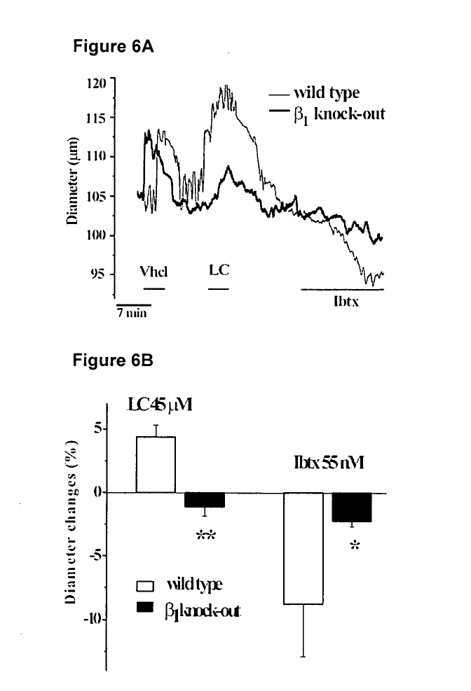

To determine the impact of LC targeting of BK β₁-subunits on organ function, LC action on the arterial diameter of pressurized cerebral arteries was evaluated from β₁-knockout vs. wt C57BU6 mice (controls). To verify the presence of functional β₁-subunit-containing BK channels in controls, artery diameter sensitivity to block by 55 nM Ibtx was tested using rat arteries (FIG. 1A). In control mice, Ibtx caused a significant vasoconstriction within 15 min of application (up to −8.7±4.2% decrease from initial diameter; p<0.01; n=4) (FIGS. 6A & 6B). As reported by Brenner et al., 2000b, Ibtx decrease in diameter was largely attenuated in arteries from β₁-knockout mice (−2.25±0.44%; different from vasoconstriction in wt mice, p<0.05; n=4) (FIGS. 6A & 6B).

The mild and transient vehicle dilation found in rat arteries was also observed in mouse arteries. Consistent with results obtained in rat arteries showing the lack of Ibtx modulation of vehicle dilation (FIG. 1A), genetic ablation of β₁-subunits failed to modify vehicle action (FIG. 6A). These results are further evidence that the observed mild and transient dilation does not involve BK channels.

More importantly, as observed in rat cerebral arteries, 45 μM LC caused a sustained yet fully reversible increase in diameter of wt mouse cerebral arteries (+4.4±0.9% from initial diameter; p<0.01; n=7). In contrast, LC consistently failed to dilate arteries from P₁-knockout mice (n=5) (FIGS. 6A & 6B), indicating that in intact cerebral arteries the presence of BK β₁-subunits is crucial for LC dilation.

Intact arteries from β₁-subunit knockout mice fail to relax in response to LC, although the arteries are able to respond to other vasoactive agents. In addition, while a variety of ion channels other than BK contribute to regulate cerebrovascular tone (Faraci and Sobey, 1998; Dietrich et al., 2005), making them putative targets of LC effect on vasomotion, the fact that genetic ablation of KCNMB1 (but not of KCNMB4) or selective pharmacological block of BK (but not other Kv) channels suppresses LC-mediated cerebrovascular dilation clearly indicates that the BK channel β₁-subunit is the molecular effector of LC-induced cerebrovascular dilation. In brief, these data identify the BK β₁-subunit as the functional target that mediates endothelium-independent LC dilation of intact and pressurized resistance arteries.

### Example 6

### Molecular Mechanism of LC Inhibition of BK Channels

The location of the molecular interaction of LC with BK channels was investigated. Experiments were conducted at physiological pH (7.35-7.4) such that the carboxylate group on the lateral chain of LC would be largely ionized. The fact that LC action on cbv1+β₁ channel Pₒ is voltage-independent (suggesting that the ionized carboxylate is not sensed across the voltage field) is consistent with the charged lateral chain residing in or nearby the aqueous solution. It is predicted that the overall hydrophobicity of the steroid nucleus will place it within the lipid bilayer. In contrast to other steroids, LC and LC analogs are planar amphiphiles. The compounds are present as a bean shape with two clear-cut “planes” or “hemispheres”. One of the hemispheres is concave and polar and the other is convex and hydrophobic. The planar polarity of the bile acid ring structure plays a role in steroid increase of BK channel Pₒ.

Data using chimeric β₁-/β₄-subunits in which the transmembrane-cytosolic ends and the extracellular loops have been swapped indicate that it is the former region that determines LC sensitivity. The β₁-subunit transmembrane regions can bring ideal interfaces for LC membrane intercalation, with the hydrophobic hemisphere of the planar amphiphile facing the bilayer lipids and the hemisphere containing the polar hydroxyl facing the β₁-subunit. In this putative model of LC location, however, the presence of polar groups on one side of the bile acid requires some polar surface to diminish the energetic cost of inserting the steroid polar groups within the hydrophobic environment of the bilayer core. Interestingly, the β₁-subunit contains an unusually high number of Thr residues in its transmembrane segments. Furthermore, β₄-subunits, which fail to sense LC, largely lack these polar residues in their transmembrane segments. Systematic mutagenesis combined with molecular modeling can be used to determine which (if any) of the polar residues present in β₁- but absent in β₄-subunits is (are) critical for interacting with bile acids.

### Example 7

### Screening Assay for Modulators of BK Channel Activity Mediated through β₁ Subunits

Functional assays to identify modulators of BK channels via beta1 subunits are performed by a multitechnical approach, similar to that used to determine LC action on BK channels and cerebrovascular tone via BK β₁ subunits. Briefly, screening assays for the modulators can be conducted by measuring vascular tone of isolated rat cerebral arteries with and without endothelium (see, for example, FIG. 1) and isolated cerebral arteries with and without endothelium obtained from wt and KCNMB1-knockout mice (see, for example, FIG. 6). In another example, screening assays for the modulators can be conducted by measuring native BK channel function in isolated cerebral artery myocytes (see, for example, FIG. 2), or by measuring function of recombinant BK channels heterologously expressed in Xenopus oocytes or mammalian HEK293 cells (see, for example, FIG. 3). Methods corresponding to these assays are provided herein above, in particular, at Examples 1 and 3.

### REFERENCES

Asher C, Wald H, Rossier B, Garty H (1996) Aldosterone-induced increase in the abundance of Na+ channel subunits. Am J Physiol 271:C605-611.
Bolotina V, Omelyanenko V, Heyes B, Ryan U, Bregestovski P (1989) Variations of membrane cholesterol alter the kinetics of Ca2(+)-dependent K+ channels and membrane fluidity in vascular smooth muscle cells. Pflugers Arch 415:262-268.
Bomzon A, Ljubuncic P (1995) Bile acids as endogenous vasodilators? Biochem Pharmacol 49:581-589.
Brenner R, Chen Q, Vilaythong A, Toney G, Noebels J, Aldrich R (2005) BK channel beta4 subunit reduces dentate gyrus excitability and protects against temporal lobe seizures. Nat Neurosci 8:1752-1759.
Brenner R, Jegla T, Wickenden A, Liu Y, Aldrich R (2000a) Cloning and characterization of novel large conductance calcium-activated potassium channel β subunits hKCNMB3 and hKCNMB4. J Biol Chem 275:6453-6461.
Brenner R, Perez G, Bonev A, Eckman D, Kosek J, Wiler S, Patterson A, Nelson M, Aldrich R (2000b) Vasoregulation by the beta1 subunit of the calcium-activated potassium channel. Nature 407:870-876.
Crowley J, Treistman S, Dopico A M (2003) Cholesterol antagonizes ethanol potentiation of human brain BKCa channels reconstituted into phospholipid bilayers. Mol Pharmacol 64:365-372.
Deenadayalu V P, White R E, Stallone J N, Gao X, Garcia A (2001) Testosterone relaxes coronary arteries by opening the large-conductance, calcium-activated potassium channel. Am J Physiol Heart Circ Physiol 281:H1720-1727.

Dick G, Sanders K (2001) (Xeno)estrogen sensitivity of smooth muscle BK channels conferred by the regulatory β₁ subunit. J Biol Chem 276:44835-44840.
Dietrich A, Mederos Y Schnitzler M, Gollasch M, Gross V, Storch U, Dubrovska G, Obst M, Yildirim E, Salanova B, Kalwa H, Essin K, Pinkenburg O, Luft F C, Gudermann T, Birnbaumer L (2005) Increased vascular smooth muscle contractility in TRPC6−/− mice. Mol Cell Biol 25:6980-6989.
Dopico A, Anantharam V, Treistman S (1998) Ethanol increases the activity of Ca2+-dependent K+ (mslo) channels: Functional interaction with cytosolic Ca2+. J Pharmacol Exp Ther 284:258-268.
Dopico A, Walsh J, Singer J (2002) Natural bile acids and synthetic analogues modulate large conductance Ca2+-activated K+ (BKca) channel activity in smooth muscle cells. J Gen Physiol 119:251-273.
Duncan R (2005) Tamoxifen alters gating of the BK α subunit and mediates enhanced interactions with the avian β subunit. Biochem Pharmacol 70:47-58.
Faraci F M, Sobey C G (1998) Role of potassium channels in regulation of cerebral vascular tone. J Cereb Blood Flow Metab 18:1047-1063.
Giangiacomo K M, Kamassah A, Harris G, McManus O (1998) Mechanism of maxi-K channel activation by dehydrosoyasaponin-I. J Gen Physiol 112:485-501.
Gourley J, Heistad D (1984) Characteristics of reactive hyperemia in the cerebral circulation. Am J Physiol 246:52-58.
Jaggar J, Li A, Parfenova H, Liu J, Umstot E, Dopico A, Leffler C (2005) Heme is a carbon monoxide receptor for large-conductance Ca2+-activated K+ channels. Circ Res 97:805-812.
Jaggar J, Porter V, Lederer W, Nelson M (2000) Calcium sparks in smooth muscle. Am J Physiol Cell Physiol 278:C235-256.
King J T, Lovell P V, Rishniw M, Kotlikoff M, Zeeman M, McCobb D (2006). Beta2 and beta4 subunits of BK channels confer differential sensitivity to acute modulation by steroid hormones. J Neurophysiol 95:2878-2888.
Knot H J, Nelson M (1998) Regulation of arterial diameter and wall [Ca²⁺] in cerebral arteries of rat by membrane potential and intravascular pressure. J Physiol 508:199-209.
Korovkina V P, Brainard A M, Ismail P, Schmidt T, England S (2004) Estradiol binding to maxi-K channels induces their down-regulation via proteasomal degradation. J Biol Chem 279:1217-1223.
Legos J, Tuma R, Barone F (2002) Pharmacological interventions for stroke: failures and future. Expert Opin Investig Drugs 11:603-614.
Liu P, Ahmed A, Jaggar J, Dopico A (2004) Essential role for smooth muscle BK channels in alcohol-induced cerebrovascular constriction. Proc Natl Acad Sci USA 101:18217-18222.
Ljubuncic P, Said O, Ehrlich Y, Meddings J, Shaffer E, Bomzon A (2000) On the in vitro vasoactivity of bile acids. Br J Pharmacol 131:387-398.
Lovell P V, King J, McCobb DP (2004) Acute modulation of adrenal chromaffin cell BK channel gating and cell excitability by glucocorticoids. J Neurophysiol 91:561-570.
Meera P, Wallner M, Jiang Z, Toro L (1996) A calcium switch for the functional coupling between a (hslo) and p subunits (Kv, Caβ) of maxi K channels. FEBS Lett 382:84-88.
Meera P, Wallner M, Toro L (2000) A neuronal β subunit (KCNMB4) makes the large conductance, voltage- and Ca2+-activated K+ channels resistant to charybdotoxin and iberiotoxin. Proc Natl Acad Sci USA 97:5562-5567.
Meredith A, Wiler S, Miller B, Takahashi J, Fodor A, Ruby N, Aldrich R (2006) BK calcium-activated potassium channels regulate circadian behavioral rhythms and pacemaker output. Nat Neurosci 9:1041-1049.
Nimigean C M, Magleby K L (2000) Functional coupling of the beta(1) subunit to the large conductance Ca(2⁺)-activated K(+) channel in the absence of Ca(2⁺). Increased Ca(2+) sensitivity from a Ca(2+)-independent mechanism. J Gen Physiol 115:719-736.
Orio P, Rojas P, Ferreira G, Latorre R (2002) New disguises for an old channel: MaxiK channel beta-subunits. News Physiol Sci 17:156-161.
Ostrow J (1993) Metabolism of bile salts in cholestasis in humans, in Hepatic Transport and Bile Secretion: Physiology and Pathophysiology (Tavoloni N and Berk P eds) pp 673-712, Raven Press, New York.
Pak J M, Lee S S (1993) Vasoactive effects of bile salts in cirrhotic rats: in vivo and in vitro studies. Hepatology 18:1175-1181.
Perez G (2005) Dual effect of tamoxifen on arterial KCa channels does not depend on the presence of the beta1 subunit. J Biol Chem 280:21739-21747.
Perez G J, Bonev A D, Nelson M T (2001) Micromolar Ca(2⁺) from sparks activates Ca(2+)-sensitive K(+) channels in rat cerebral artery smooth muscle. Am J Physiol Cell Physiol 281:C1769-1775.
Roda A, Cerre C, Fini A, Sipahi A, Baraldini M (1995) Experimental evaluation of a model for predicting micellar composition and concentration of monomeric species in bile salt binary mixtures. J Pharm Sci 84:593-598.
Thibault N, Ballet F (1993) Effect of bile acids on intracellular calcium in isolated rat hepatocyte couplets. Bioch Pharm 45:289-293.
Valverde M A, Rojas P, Amigo J. Cosmelli D, Orio P, Bahamonde M I, Mann G E, Vergara C, Latorre R (1999) Acute activation of Maxi-K channels (hSlo) by estradiol binding to the beta subunit. Science 285:1929-1931.
Watson C S, Gametchu B (2003) Proteins of multiple classes may participate in nongenomic steroid actions. Exp Biol Med (Maywood) 228:1272-1281.
White R, Han G, Maunz M, Dimitropoulou C, El-MowafyA, Barlow R, Catravas J, Snead C, Carrier G, Zhu S, Yu X (2002) Endothelium-independent effect of estrogen on Ca(2+)-activated K(+) channels in human coronary artery smooth muscle cells. Cardiovasc Res 53:650-661.
Williams C A, Sheppard T, Marrufo M, Galbis-Reig D, Gaskill A (2003). A brief descriptive analysis of stroke features in a population of patients from a large urban hospital in Richmond, Va., a city within the ‘stroke belt’. Neuroepidemiology 22:31-36.

It will be understood that various details of the presently disclosed subject matter may be changed without departing from the scope of the presently disclosed subject matter. Furthermore, the foregoing description is for the purpose of illustration only, and not for the purpose of limitation.

1. A method of screening candidate compositions for an ability to modulate myocyte BK channel activity, the method comprising:
(a) establishing a test sample comprising a myocyte BK channel β₁ subunit; and
(b) measuring the interaction, effect, or combination thereof, of a candidate composition on the test sample to thereby determine the ability of the candidate composition to modulate myocyte BK channel activity.

2. The method of claim 1, wherein the candidate composition comprises a lithocholate, or an analog or derivative thereof.

3. The method of claim 2, wherein the myocyte is a small resistance artery myocyte.

4. A method of screening for candidate compositions useful in the treatment of a disorder where increasing myocyte BK channel activity can attenuate, revert or prevent the disorder, the method comprising:
(a) establishing a test sample comprising a myocyte BK channel β₁ subunit; and
(b) measuring the interaction, effect, or combination thereof, of a candidate composition on the test sample to thereby determine the ability of the candidate composition to increase myocyte BK channel activity and attenuate, revert or prevent the disorder.

5. The method of claim 4, wherein the candidate composition comprises a lithocholate, or an analog or derivative thereof.

6. The method of claim 4, wherein the myocyte is a small resistance artery myocyte.

7. The method of claim 4, wherein the attenuation, reversion or prevention of the disorder is mediated at least in part through modulation of blood pressure and/or blood flow.

8. The method of claim 4, wherein the attenuation, reversion or prevention of the disorder is mediated at least in part through vasodilation.

9. The method of claim 8, wherein the vasodilation is characterized by an increase in blood vessel diameter of about 10 percent.

10. A method of treating a subject having a disorder, the method comprising, administering to the subject a composition comprising a compound capable of increasing myocyte BK channel activity mediated by the BK channel by subunit, wherein increasing myocyte BK channel activity can attenuate, revert or prevent the disorder.

11. The method of claim 10, wherein the compound comprises a lithocholate, or an analog or derivative thereof.

12. The method of claim 10, wherein the administration of the composition comprising the compound capable of increasing myocyte BK channel activity results in decreases in blood pressure and/or increases in blood flow.

13. The method of claim 12, wherein the increase in blood flow is mediated at least in part through vasodilation.

14. The method of claim 13, wherein the vasodilation occurs at least in part in small resistance arteries.

15. The method of claim 14, wherein the small resistance arteries are small cerebral arteries.

16. The method of claim 15, wherein the vasodilation in the small cerebral arteries is about a 10 percent increase in arterial diameter.

17. The method of claim 16, wherein the increase in blood flow in the small cerebral arteries is about 30 percent.

18. A pharmaceutical composition useful for increasing myocyte BK channel activity in a subject, wherein the composition comprises an effective amount of a lithocholate or an analog or derivative thereof; and a pharmaceutically acceptable carrier.

19. A pharmaceutical composition useful for increasing myocyte BK channel activity in a subject, wherein the BK channel activity is mediated by the BK channel β₁ subunit.

20. A method of selectively targeting tissues comprising BK channel β₁ subunits in a subject, the method comprising administering to the subject a composition comprising a compound capable of selectively binding myocyte BK channel β₁ subunits, whereby tissues comprising BK channel β₁ subunits are targeted.

21. The method of claim 20, wherein the composition comprises a lithocholate, or an analog or derivative thereof.

22. The method of claim 20, wherein the tissues targeted are smooth muscle tissues.

23. The method of claim 20, wherein the composition further comprises an imaging agent.

24. The method of claim 20, wherein the composition further comprises a pharmaceutically acceptable carrier.

25. The method of claim 20, wherein the subject is a mammal.
